(19) United States
(12) Patent Application Publication
(10) Pub. No.: US 2023/0178195 A1
(43) Pub. Date: Jun. 8, 2023

(54) System, Method, and Software for Managing Digital Animal Records

(71) Applicant: Prox Animal Health, LLC, Fort Worth, TX (US)

(72) Inventor: Barry Beverly, Fort Worth, TX (US)

(21) Appl. No.: 18/061,439

(22) Filed: Dec. 3, 2022

Related U.S. Application Data
(60) Provisional application No. 63/264,918, filed on Dec. 3, 2021.

(51) Int. Cl. G16H 10/60

(52) U.S. Cl. CPC G16H 10/60

(57) ABSTRACT

In certain embodiments, a method includes accessing, in response to a first user input and via a storage system, digital animal records that include animal data (including inventory and health data) for animals; and generating, according to the inventory data, an inventory user interface (UI) that includes identifying information of a first portion of the animals. The method includes accessing, in response to a second user input and via the storage system, the health data and generating, according to the health data, a health records UI for managing the health records and that includes medical records of a second portion of the animals. The method includes receiving, via the inventory UI, inventory update data and, in response, transmitting an inventory update to the storage system. The method includes receiving, via the health records UI, health records update data and, in response, transmitting a health records update to the storage system.

### CROSS-REFERENCE TO RELATED APPLICATIONS

This application claims the benefit of U.S. Provisional Application No. 63/264,918, filed on Dec. 3, 2021, which is incorporated by reference.

### TECHNICAL FIELD

This disclosure relates generally to software, and, in particular embodiments, to a system, method, and software for managing digital animal records.

### BACKGROUND

Farms, ranches, and the like often maintain hundreds if not thousands of animals for a variety of purposes. For example, family or corporate livestock operations may raise cows, pigs, chickens, horses, and/or other animals for one or more commercial purposes. In one example, the commercial purpose may include raising the animals to sell as a meat food source. In another example, the commercial purpose may include raising cattle as a source of dairy products. As part of raising the animals, workers associated with the farm may be responsible for keeping track of the animals and managing the health of the animals being raised on the farm. Managing the health of the animals may include managing the vaccinations given to the animals, managing any treatments given to the animals, managing the breeding of the female animals, and other suitable tasks.

### SUMMARY

In certain embodiments, a non-transitory computer-readable storage medium stores a program for execution by one or more processors. The program includes instructions for accessing, in response to a first user input and via an animal records storage system, digital animal records for animals. The digital animal records include animal data for the animals, the animal data including animal inventory data and animal health data. The program includes instructions for generating, according to the animal inventory data, an animal inventory user interface that includes identifying information of at least a first portion of the animals. The program includes instructions for accessing, in response to a second user input and via the animal records storage system, the animal health data for the animals and generating, according to the animal health data, an animal health records user interface for managing the animal health records of the animals. The animal health records user interface includes medical records of at least a second portion of the animals. The program includes instructions for receiving, via the animal inventory user interface, animal inventory update data and transmitting, in response to the animal inventory update data, an animal inventory update to the animal records storage system for updating the animal inventory data. The program includes instructions for receiving, via the animal health records user interface, animal health records update data and transmitting, in response to the animal health records update data, an animal health records update to the animal records storage system for updating the animal health data.

In certain embodiments, a system includes a storage system, one or more processors, and a non-transitory computer-readable storage medium storing a program to be executed by the one or more processors. The program includes instructions for receiving animal data for animals, the animal data including animal inventory data and animal health data, and for generating, in response to receiving the animal data, digital animal records on the storage system, the digital animal records including the animal data. The program includes instructions for receiving, from a first requesting device, a request for the animal inventory data for the animals; accessing, from digital animal records on the storage system, the animal inventory data for the animals; and transmitting, to the first requesting device, the animal inventory data for display by an application on the first requesting device. The program includes instructions for receiving, from a second requesting device, a request for at least a portion of the animal health data for a subset of the animals; accessing, from the digital animal records on the storage system, the animal health data for the subset of the animals; and transmitting, to the second requesting device, the animal health data for the subset of the animals for display by an application on the second requesting device.

In certain embodiments, a method includes receiving animal data for animals that includes inventory data and health data; and generating, in response to receiving the animal data, digital animal records that include the animal data on a storage system. The method includes receiving, from a first device, a request for the inventory data; accessing, from the digital animal records, the inventory data; and transmitting, to the first device, the inventory data for display by an application on the first device. The method further includes receiving, from a second device, a request for at least a portion of the health data for a subset of the animals; accessing, from the digital animal records, the health data for the subset; and transmitting, to the second device, the health data for the subset for display by an application on the second device.

In certain embodiments, a method includes accessing, in response to a first user input and via a storage system, digital animal records that include animal data (including inventory and health data) for animals; and generating, according to the inventory data, an inventory user interface (UI) that includes identifying information of a first portion of the animals. The method includes accessing, in response to a second user input and via the storage system, the health data and generating, according to the health data, a health records UI for managing the health records and that includes medical records of a second portion of the animals. The method includes receiving, via the inventory UI, inventory update data and, in response, transmitting an inventory update to the storage system. The method includes receiving, via the health records UI, health records update data and, in response, transmitting a health records update to the storage system.

### BRIEF DESCRIPTION OF THE DRAWINGS

For a more complete understanding of this disclosure, and the advantages thereof, reference is now made to the following descriptions taken in conjunction with the accompanying drawings, in which:

FIG. 1 illustrates an example system for managing digital animal records for managing animal health, according to certain embodiments;

FIG. 2 illustrates an example user device for managing digital animal records for managing animal health, according to certain embodiments;

FIG. 3 illustrates an example processing system for managing digital animal records for managing animal health, according to certain embodiments;

FIGS. 4A-4L illustrate example user interfaces that may be generated for managing animal health, according to certain embodiments;

FIG. 5 illustrates example digital animal records, according to certain embodiments;

FIG. 6 illustrates an example method for managing digital animal records for managing animal health, according to certain embodiments; and

FIG. 7 illustrates an example method for managing digital animal records for managing animal health, according to certain embodiments.

### DETAILED DESCRIPTION OF ILLUSTRATIVE EMBODIMENTS

Managing the inventory and health of animals being raised for food or other purposes is a daunting task. Conventionally, the vast majority of inventory records and animal health procedures and records have been recorded by hand by farmers or other workers in the field, using a notepad for example, which may be not only unreliable, inefficient, and insecure, but may lack an ability for the health record to travel with an animal, either to a show or when it is sold, or otherwise be accessible in a variety of locations. For example, commercial beef and swine farms still use paper records, which is highly inefficient. Paper records may be inefficient on staffing and productivity, and may provide no practical way to see disease outbreak trends. Producers may lose track of animal health events and illness, potentially increasing costs. Furthermore, with paper records, no electronic medical record exists that can be shared in a digital format. As another example, relying on printed reports within a company (e.g., the company operating the animal farm) may lack an ability to perform/review immediate data analytics. As another example, relying on printed reports within a company (e.g., the company operating the animal farm) may lack an ability to generate/view health outcomes data or drug withdrawal data (e.g., as it relates to the meat processing flow).

Furthermore, even to the extent some or all of this information could be entered into an electronic spreadsheet, interacting with and managing a spreadsheet, let alone on a scale that might be associated with certain types of use cases, would be inconvenient, difficult, error-prone, and frustrating. Spreadsheets quickly become unmanageable and lack any intuitive interface for visualizing and interacting with the data. Furthermore, interacting with a spreadsheet can present other problems, such as access control, version management, and the like.

Embodiments of this disclosure provide a livestock animal health management software platform, which may promote animal health in cattle (e.g., beef or dairy cattle), swine, sheep, or other types of animals/livestock. The software platform may be implemented, at least in part, as a software application (e.g., a mobile application or other suitable type of software application). At least a portion of the system may be implemented as a mobile application that can be downloaded by a user to a user device (e.g., a mobile device) of the user, such as a mobile smartphone or tablet computer. Users may include any suitable types of users, such as farmers/ranchers and associated users (e.g., employees or otherwise authorized users), veterinarians, or other suitable users.

Embodiments of this disclosure provide a server system for generating and maintaining a database of inventory records and health records for a plurality of animals.

Embodiments of this disclosure provide an application for executing on user devices and that is configured to generate user interfaces for entering, accessing, and displaying animal data, including animal inventory data, animal health data, and the like. The user interfaces may facilitate entering and receiving data for updating the animal health records, and for sending the updates to data storage for maintaining a centralized digital health record for the animal. The user interface screens may provide a highly intuitive arrangement for accessing, managing, and viewing the animal health records.

The architecture described herein may be implemented in a computer or network of computers having one or more processors executing instructions of software programs that are stored in one or more computer-readable storage.

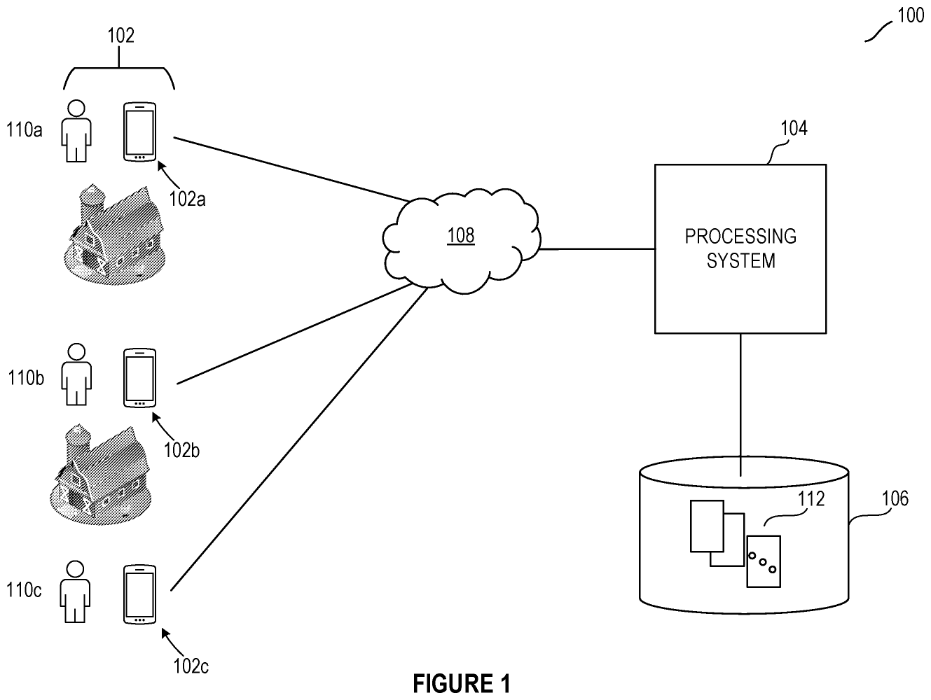

FIG. 1 illustrates an example system 100 for managing digital animal records for managing animal health, according to certain embodiments. In the illustrated example, system 100 includes user devices 102, processing system 104, storage unit 106, and network 108. In general, system 100 may be used to manage health of animals using digital animal records stored on storage unit 106 and via a software application installed on user devices 102.

User devices 102 may include any suitable computing device(s) capable of running software applications and accessing network 108. User devices 102 may be implemented in any suitable combination of hardware, firmware, and software. User devices 102 may include one or more computer systems at one or more locations. Each computer system may include any appropriate input devices, output devices, mass storage media, processors, memory, or other suitable components for receiving, processing, storing, and communicating data. For example, each computer system may include an integrated circuit (IC), printed circuit board (PCB), personal computer, laptop computer, workstation, network computer, kiosk, wireless data port, personal data assistant (PDA), one or more Internet Protocol (IP) telephones, one or more cellular/smart phones, tablet computers, wearable devices (e.g., smartwatch), one or more processors within these or other devices, or any other suitable processing device. Certain ones of the above or other user devices 102 may be mobile devices, such that they may be readily hand-carried by a user.

Users 110a-110c (referred to generally as users no) of user devices 102 may include any suitable users. In certain embodiments, users no of user devices 102 may include farmers, employees of a farm or other livestock operation, veterinarians, or any other suitable users who may access or otherwise interact with digital animal records. In certain embodiments, at least a portion of users no of user devices 102 include personnel associated with raising animals. In the illustrated example, user 110a of user device iota and user 110b of user device 102b represent users present on a farm who interact with the animals and access and otherwise interact with digital animal records via user devices iota and 102b. User 110c of user device 102C represents a user who may be remote from the farm but still be able to access and otherwise interact with digital animal records 112 via user device 102C. Users 110a-110c may be associated with the same farms or different farms, and those farms may raise the same or different types of animals. Furthermore, the farms might or might not be associated with one another from an ownership/management perspective.

Using user devices 102, users no may enter, access, modify, filter, and otherwise interact with digital animal records 112 stored by processing system 104. Software running on user devices 102 may provide a variety of functionality, including generating and displaying associated user interfaces, to allow users no of user devices 102 to enter, access, modify, filter, and otherwise interact with digital animal records 112 stored by processing system 104.

For example, user devices 102 may allow users no to enter animal data for animals to be managed using system 100, and to create digital animal records 112 to be stored by processing system 104. The animal data may include animal inventory data and/or animal health data. For example, users no of user devices 102 may enter various biographical information about the animals to be managed using system 100, such as an identifier, a sex, a birthdate, location information, or any other suitable information that may be useful in maintaining an inventory of animals to be managed using system 100. As another example, users no of user devices 102 may enter various health data about the animals to be managed using system 100, such as medical records, vaccination dates, health events, treatments, breeding records, or any other suitable health data that may be useful in maintaining the health of an inventory of animals to be managed using system 100.

As another example, using user devices 102 users no may search and filter digital animal records 112. For example, using user devices 102, users no may be able to access various user interface screens that provide views into different portions of the digital animal records 112. As a particular example, using user devices 102, users no may be able to view the entire population of animals being managed using system 100, filter down to a particular subset of animals being managed using system wo (e.g., view certain information about the animals in a particular location), or view particular records for particular animals.

As another example, using user devices 102, users no may be able to set reminders for particular health events for the animals. For example, users no may be able to set reminders to administer particular vaccines or medications, schedule veterinary visits, inseminate female animals, and the like.

As another example, using user devices 102 users no may be able to manage the breeding of the female population of the animals being managed using system mo. For example, user devices 102 may allow users to maintain breeding schedules, view historical breeding records, manage embryo transfer records, view breeding records, set reminders associated with breeding, or perform other suitable tasks associated with managing the breeding of the female portion of the managed animal population.

As another example, using user devices 102 users no may be able to upload, view, and manage documentation associated the animals being managed using system 100. Such documentation may include birth certificates, vaccination records, veterinary visit summaries, or any other suitable documentation. In certain embodiments, users no may be able to upload videos, such as videos of veterinary visits.

As another example, using user devices 102 users no may be able to manage health events associated with the managed animal population. For example, users no may be able to record and track illnesses, track administration of treatments for those illnesses, recognize and follow trends to detect an outbreak of certain illnesses, and the like.

As another example, using user devices 102 users no may be able to enter and record the death of animals in the managed animal population. For example, users no may be able to view mortality reports for the managed animal population.

Example features that may be provided using user devices 102 may include digital electronic animal health records, digital electronic medical records, breeding records, embryo transfer records, veterinarian video chat, vaccination and medical reminders, an away care portal, a weather prediction application, digital animal inventory, dashboard analytics, and security. Certain embodiments allow a user to view, share, and/or update vaccination, medication (e.g., antibiotic), and dewormer records and reminders. Certain embodiments allow a user to view, share, and/or update photo-enhanced herd health, breeding, and heat cycle records. Certain embodiments allow a user (e.g., a farmer) to connect with a veterinarian, including potentially after normal business hours, via a video chat within the application. Certain embodiments allow users to share X-rays (e.g., a farmer to share an X-ray with a veterinarian). Certain embodiments allow users to coordinate pasture and grass management. Certain embodiments facilitate maintaining farrier and dental health records.

Certain embodiments provide a dashboard feature. The dashboard feature may include a digital analytics dashboard showcasing animal health and management using real-time data. The dashboard may showcase the animal health/veterinary procedures, potentially for the entire animal inventory being managed using system wo (e.g., the entire pig herd). The data displayed in the dashboard may include vaccinations, dewormers, antibiotics, and/or any infections/disorders encountered. An animal health portal, which may be organized by lot or other subdivision if appropriate, may fit elegantly into a corporate animal farm's operation, possibly providing the ability to view animal health records (e.g., vaccinations, dewormers, antibiotics, and medications), organized by or other subdivision lot if appropriate. Certain embodiments may include a digital electronic medical record for all animals (e.g., all pigs, market hogs, and sows), which also may be synchronized to a veterinary department of the organization. Production records may be synchronized and viewed by the lot format. In certain embodiments, if desired, production records can be viewed by all departments within the company. Certain embodiments reduce or eliminate paper records, transferring to completely digital format and producing an individual electronic medical record for each animal. Certain embodiments may be more biosecurity-compliant by reducing the number of off-farm visitors who could potentially put the animals at greater risk. Certain embodiments provide antibiotic withdrawal data analytics in a digital format, which may be synchronized to one or more meat processing plants. Certain embodiments may reduce complicated state and federal audits due to a digital record platform.

Processing system 104 may include any suitable computing device(s) operable to communicate with one or more user devices 102 through network 108 to facilitate managing animal health. Processing system 104 may be implemented in any suitable combination of hardware, firmware, and software. Processing system 104 may include one or more computer systems (e.g., one or more servers) at one or more locations. Each computer system may include any appropriate input devices, output devices, mass storage media, processors, memory, or other suitable components for receiving, processing, storing, and communicating data. Processing system 104 may include, for example, a file server, a domain name server, a proxy server, a web server, a computer workstation, or any other device operable to respond to requests for data from user devices 102 and/or manage storage and processing of data in storage unit 106. Processing system 104 may execute with any of the well-known MS-DOS, PC-DOS, OS-2, MAC-OS, WINDOWS, UNIX, or other appropriate operating systems, including future operating systems.

Processing system 104 may include one or more software applications that communicate with a software application running on user devices 102 and that facilitate creating, maintaining, and searching of digital animal records 112 in storage unit 106.

Storage unit 106 may include any number of tangible storage media. Additionally, all or part of storage unit 106 could reside locally within processing system 104 or could reside at a location external but accessible to processing system 104 (e.g., within removable media, and/or at an external database). In certain embodiments, storage unit 106 may take the form of a suitable combination of volatile and non-volatile memory including, without limitation, magnetic media, optical media, random access memory (RAM), read only memory (ROM), removable media, or any other suitable memory component. Where appropriate, RAM may be dynamic RAM (DRAM) or static RAM (SRAM). Moreover, where appropriate, this RAM may be single-ported or multi-ported RAM. This disclosure contemplates any suitable RAM. In certain embodiments, storage unit 106 may include one or more databases. In certain embodiments, a portion of all of storage unit 106 may include a database, such as one or more structured query language (SQL) servers or relational databases. In certain embodiments, a portion or all of storage unit 106 may implement a portion or all of a blockchain. The one or more storage units 106 may be implemented using one or more computer systems/servers and may be connected to a network via which user devices may be able to access/update associated data records.

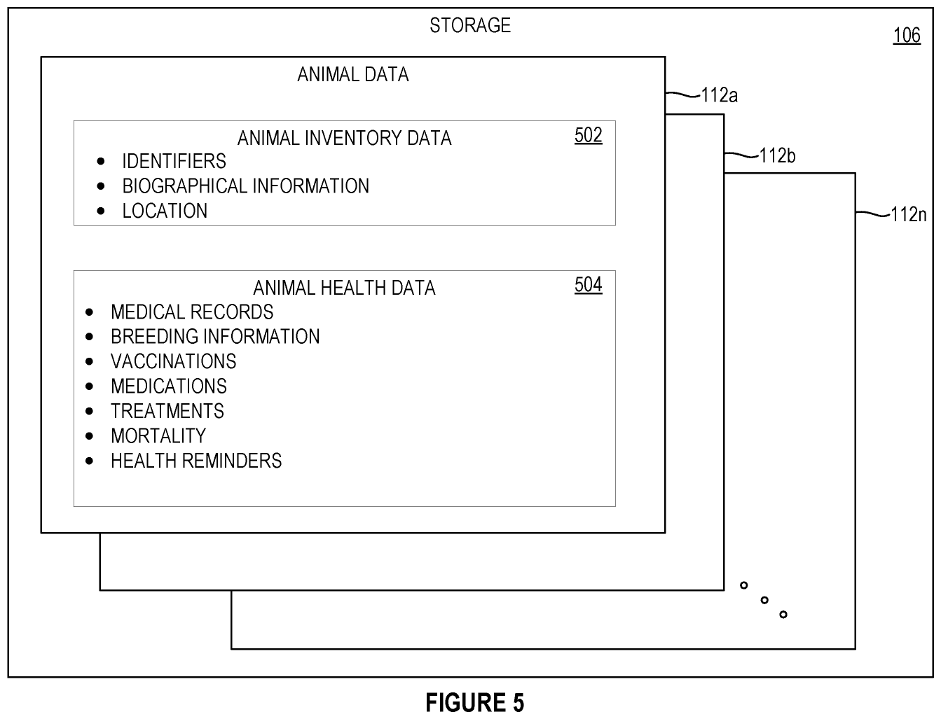

Storage unit 106 may be used to store digital animal records 112 that contain information that facilitates managing animal health of a plurality of animals. In certain embodiments, digital animal records 112 may include animal data, which may include animal inventory data, animal health data, any combination of the preceding, or other data that may facilitate managing animal health of a plurality of animals. Additional detail regarding digital animal records 112 is described below with reference to FIG. 5.

As just one example, embodiments may store collected data on one or more networked storage devices (e.g., in the so-called cloud). Certain embodiments use human-grade Health Insurance Portability and Accountability Act of 1996 (HIPAA)-compliant security for privacy. In certain embodiments, both users associated with a farm and one or more authorized veterinarians may be able to update/access at least some of the same data records as stored on the network so that such users are viewing or otherwise interacting with synchronized data.

User devices 102 may access features provided by processing system 104 and/or digital animal records 112 stored using storage unit 106 via network 108. Network 108 may include wireless and/or wireline platforms operable to exchange electronic data or information. In certain embodiments, network 108 may include one or more packet data networks offering communication interfaces or exchanges between components of system 100. Network 108 include any suitable combination of local area networks (LANs), metropolitan area networks (MANs), wide area networks (WANs), wireless local area networks (WLANs), virtual private networks (VPNs), intranets, Internets, plain old telephone systems (POTSs), or any other appropriate architecture or system that facilitates electronic communications. Certain networks 108 include wireless technology such as WiMax (802.16), WiFi (802.11), 3G, 4G, 5G, or any other suitable wireless technologies in any suitable combination.

Although particular animals may be described, this disclosure contemplate using any of the described features being used for any suitable type of animal/farm. For example, a cow application may be implemented having one or more features similar to those described herein with reference to the swine application.

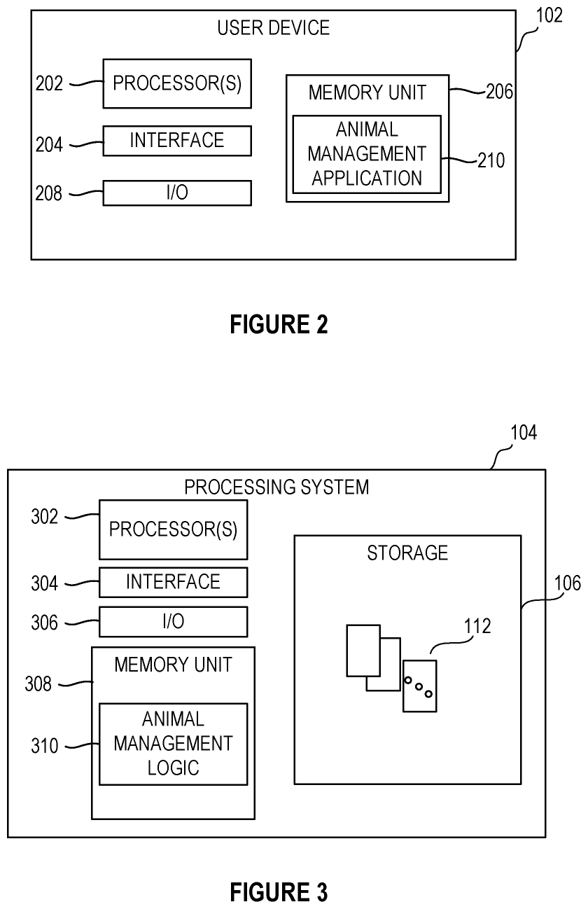

FIG. 2 illustrates an example user device 102, according to certain embodiments. In the illustrated example, user device 102 includes one or more processors 202, one or more interfaces 204, one or more memory units 206, and various I/O functionality 208, each referred to hereinafter in the singular for simplicity. In certain embodiments, user device 102 includes a combination of hardware, firmware, and software that facilitates managing animal health.

Processor 202 may refer, for example, to the one or more tangible devices of user device 102 capable of carrying out instructions, such as that of a computer program. For example, processor 202 may include one or more microprocessors, controllers, or any other suitable computing devices or resources. Processor 202 may work, either alone or with other components of user device 102, to provide a portion or all of the functionality of user device 102 described herein. For example, processor 202 may be execute or realize various logic-based functions of animal management application 210.

Interface 204 may include, for example, any tangible device(s) that may be used (e.g., by a person, or by another device or system) to communicate with user device 102. For example, any suitable combination of keypads, touch screens, and speech recognition devices may be considered input interfaces 204 and a display screen may be considered output interfaces 204.

Memory unit 206 may include any number of tangible storage media. Additionally, all or part of memory unit 206 could reside locally within user device 102 or could reside at a location external but accessible to user device 102 (e.g., within removable media, and/or at an external database). In certain embodiments, memory unit 206 may take the form of a suitable combination of volatile and non-volatile memory including, without limitation, magnetic media, optical media, RAM, ROM, removable media, or any other suitable memory component. Where appropriate, RAM may be DRAM or SRAM. This disclosure contemplates any suitable RAM. Memory unit 206 may include, for example, applications programs, network, communication programs, operating system software data, etc.

I/O functionality 208 may include, for example, any communication method of user device 102 (e.g., from an image buffer to a display). Input functionality may include, for example, the receiving of signals or data by user device 102. Output functionality may include, for example, the communication of signals or data by user device 102. These terms may also refer to part of an action, such as, for example, to “perform I/O” may involve performing an input and/or output operation. In some cases, one or more communication devices of user device 102 may be used to implement both input and output functionality.

User device 102 may include, or may be configured to access, animal management application 210 that when executed facilitates managing animal health. Animal management application 210 may be implemented in any suitable combination of hardware, firmware, and software. In certain embodiments, animal management application 210 includes a set of computer-readable instructions (e.g., stored in memory unit 206 or some other suitable computer-readable storage medium) that when executed by processor 202 are operable to perform certain operations.

Animal management application 210 may generate user interfaces and provide associated functionality for managing digital animal records 112.

Animal management application 210 may be web-based, for example, with parts of each page being loaded from a server and/or a database, or may be natively-compiled to execute on user device 102 even when no network is available. Although primarily described as a software application running on user device 102, this disclosure contemplates animal management application 210 being implemented as a set of web pages accessible in a web browser, for example.

In operation of an example embodiment of user device 102, animal management application 210 may access, in response to user input and via an animal records storage system (e.g., storage unit 106), digital animal records 112 for a plurality of animals. Digital animal records 112 may store animal data for the animals, and the animal data may include animal inventory data and animal health data.

Animal management application 210 may generate, according to the animal inventory data, an animal inventory user interface. The animal inventory user interface may include identifying information of at least a first portion of the animals being managed using system boo. In certain embodiments, animal management application 210 may receive, via the animal inventory user interface, a selection of a subset of the animals and generate an updated animal inventory user interface according to the selection of the subset of animals. For example, using one or more filter fields, a user may specify to view some or all of the animal inventory data for a particular subset of the animals. In response to the filter request, animal management application 210 may communicate with animal management logic of processing system 104 (e.g., animal management logic 310, described below) to retrieve the requested animal inventory data and display an animal inventory user interface that includes the requested animal inventory data.

In certain embodiments, the animal inventory user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

Animal management application 210 may access, in response to user input using user device 102 and via the animal records storage system (e.g., storage unit 106), animal health data for the animals. Animal management application 210 may generate, according to the animal health data, an animal health records user interface for managing the animal health records of the animals. The animal health records user interface may include medical records of at least a portion of the animals.

Animal management application 210 may receive, via the animal inventory user interface displayed on user device 102, animal inventory update data, and animal management application 210 may transmit, in response to the animal inventory update data received via the animal inventory user interface, an animal inventory update to the animal records storage system (e.g., storage unit 106) for updating digital animal records 112 (e.g., animal inventory data).

Animal management application 210 may receive, via the animal health records user interface displayed on user device 102, animal health records update data. Animal management application 210 may transmit, in response to the animal health records update data received via the animal health records user interface, an animal health records update to the animal records storage system (e.g., storage unit 106) for updating digital animal records 112 (e.g., animal health data).

In certain embodiments, animal management application 210 may generate a dashboard user interface screen that includes selectable links. Each selectable link may correspond to an associated feature and data set for managing animal health of the managed animal inventory. For example, a first selectable link may include a link to the animal inventory user interface. As another example, a second selectable link may include a link to a breeding records user interface. As another example, a third selectable link may include a link to a medical records user interface. As another example, a fourth selectable link may include a link to a health reminders user interface. As another example, a fifth selectable link may include a link to a treatment user interface. As another example, a sixth selectable link may include a link to a mortality report user interface.

In certain embodiments, animal management application 210 is configured to access, via the animal records storage system (e.g., storage unit 106), breeding information for a subset of the animals, and to generating, according to the breeding information, a breeding user interface. As examples, the breeding user interface may include breeding information for the subset of animals or the breeding user interface may include breeding details for a particular animal of the subset of animals.

In certain embodiments, animal management application 210 is configured to access, via the animal records storage system (e.g., storage unit 106), mortality information of the animals, and to generate, according to the mortality information, a mortality user interface summarizing the mortality information for the animals.

FIG. 3 illustrates an example processing system 104 and associated storage unit 106, according to certain embodiments. In the illustrated example, processing system 104 includes one or more processors 302, one or more interfaces 304, various I/O functionality 306, one or more memory units 308, and storage unit 106, each referred to hereinafter in the singular for simplicity. In certain embodiments, processing system 104 includes a combination of hardware, firmware, and software that facilitates managing animal health.

Processor 302 may refer, for example, to the one or more tangible devices of processing system 104 capable of carrying out instructions, such as that of a computer program. For example, processor 302 may include one or more microprocessors, controllers, or any other suitable computing devices or resources. Processor 302 may work, either alone or with other components of processing system 104, to provide a portion or all of the functionality of processing system 104 described herein. For example, processor 302 may execute or realize various logic-based functions of animal management logic 310.

Interface 304 may include, for example, any tangible device(s) that may be used (e.g., by a person, or by another device or system) to communicate with processing system 104. In certain embodiments, any suitable combination of keyboards, mice, touch screens, and speech recognition devices may be considered an input interfaces 304 and monitors and printers may be considered output interfaces 304.

I/O functionality 306 may include, for example, any communication method of processing system 104 (e.g., from an image buffer to a display). Input functionality may include, for example, the receiving signals or data by processing system 104. Output functionality may include, for example, the communication of signals or data by processing system 104. These terms may also refer to part of an action, such as, for example, to “perform I/O” may involve performing an input and/or output operation. In some cases, one or more communication devices of processing system 104 may be used to implement both input and output functionality.

Memory unit 308 may include any number of tangible storage media. Additionally, all or part of memory unit 308 could reside locally within processing system 104 or could reside at a location external but accessible to processing system 104 (e.g., within removable media, and/or at an external database). In certain embodiments, memory unit 308 may take the form of a suitable combination of volatile and non-volatile memory including, without limitation, magnetic media, optical media, RAM, ROM, removable media, or any other suitable memory component. Where appropriate, RAM may be DRAM or SRAM. This disclosure contemplates any suitable RAM. Memory unit 308 may include, for example, applications programs, network, communication programs, operating system software data, etc.

Processing system 104 may include, or may be configured to access, animal management logic 310 that when executed facilitates managing animal health. Animal management logic 310 may be implemented in any suitable combination of hardware, firmware, and software. In certain embodiments, animal management logic 310 includes a set of computer-readable instructions (e.g., stored in memory unit 308 or some other suitable computer-readable storage medium) that when executed by processor 302 are operable to perform certain operations. In this example, animal management logic 310 is stored within memory unit 308; however, animal management logic 310 may be stored external to memory unit 308 and/or external to processing system 104.

Animal management logic 310 is an application that communicates with animal management applications 210 on user devices 102 and that facilitates creating, maintaining, and searching of digital animal records 112 in storage unit 106.

In certain embodiments, animal management logic 310 may enforce one or more security measures for restricting access, potentially with different tiers of access and operation capabilities, to digital animal records 112 stored on storage unit 106. Thus, users no may register with processing system 104, and animal management logic 310 of processing system 104 may manage the rights of those users to view (or not view), edit (or not edit), or otherwise interact with digital animal records 112.

The description of storage unit 106 from FIG. 1 is incorporated by reference. Additionally, all or part of storage unit 106 could reside locally within processing system 104 or could reside at a location external but accessible to processing system 104 (e.g., within removable media, and/or at an external database).

As described above, storage unit 106 may be used to store digital animal records 112 that contain data for managing animal health. In certain embodiments, digital animal records no may include animal data, which may include animal inventory data, animal health data, any combination of the preceding, or other information that may facilitate managing animal health. For example, the animal inventory data may include various biographical information about the source animals, such as an identifier, a sex, a birthdate, location information, or any other suitable information that may be useful in maintaining an inventory of source animals. As another example, health data about the source animals may include medical records, vaccination dates, health events, treatments, breeding records, or any other suitable health information. Additional detail regarding example digital animal records no are described below with reference to FIG. 5.

In certain embodiments, the described processing and memory elements (such as processor(s) 302, memory unit 308, and storage unit 106) may be distributed across multiple devices such that the operations performed utilizing these elements may also be distributed across multiple devices. For example, software operated utilizing these elements may be run across multiple computers that contain these processing and memory elements. Other variations aside from the stated example are contemplated involving the use of distributed computing.

In operation of an example embodiment of processing system 104, animal management logic 310 may receive animal data for multiple animals. The animal data may include animal inventory data and animal health data. Animal management logic 310 may generate, in response to receiving the animal data for the animals, digital animal records 112 on the storage system (e.g., storage unit 106). Digital animal records 112 may include the animal data for the animals.

Animal management logic 310 may receive, from a requesting user device 102, a request for the animal inventory data for the animals. Animal management logic 310 may access, from digital animal records 112 on the storage system (e.g., storage unit 106), the animal inventory data for the animals. Animal management logic 310 may transmit, to the requesting user device 102, the animal inventory data for display by an application (e.g., animal management application 210) running on the requesting user device 102. In certain embodiments, the animal inventory data includes the following information for the managed inventory of animals: an animal identifier; animal type; and location information.

Animal management logic 310 may receive, from a requesting user device 102, a request for at least a portion of the animal health data for a subset of the animals. Animal management logic 310 may access, from digital animal records 112 on the storage system (e.g., storage unit 106), the animal health data for the subset of the animals. Animal management logic 310 may transmit, to the requesting user device 102, the animal health data for the subset of the animals for display by an application (e.g., animal management application 210) running on the requesting user device 102. In certain embodiments, the animal health data includes the following information for the managed inventory of animals: digital medical records; vaccination information; breeding information; treatment information; and mortality information. In certain embodiments, the animal health data includes a schedule of health events and animal management logic 310 is configured to communicate a reminder of a health event to an application (e.g., animal management application 210) running on a requesting user device 102.

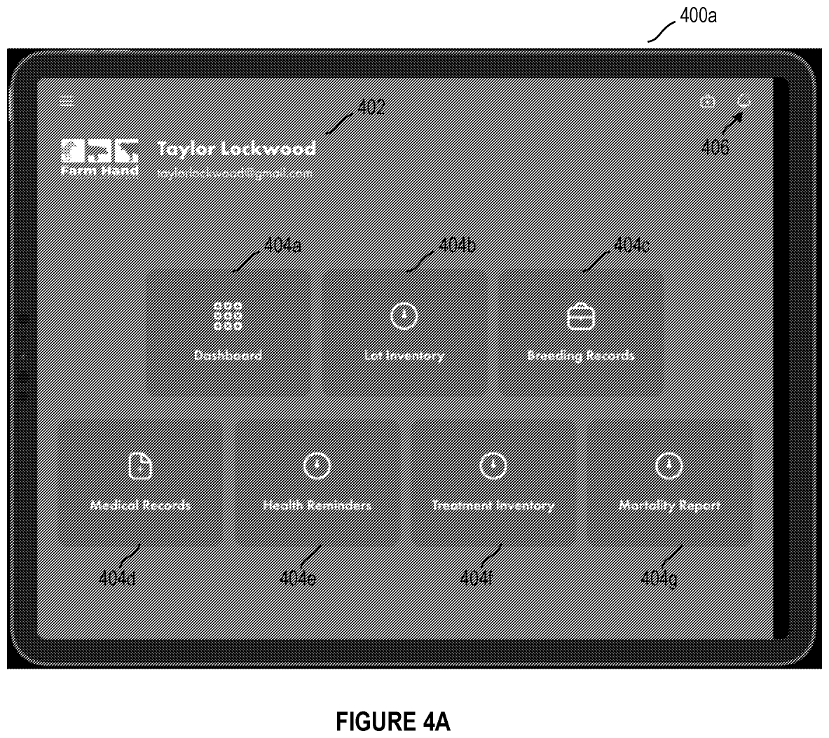

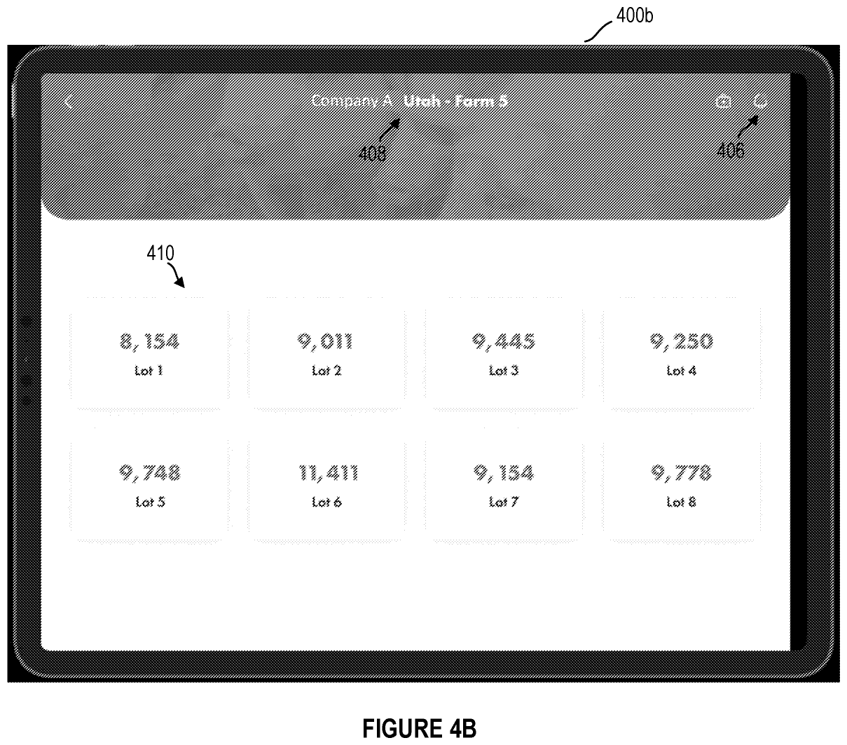

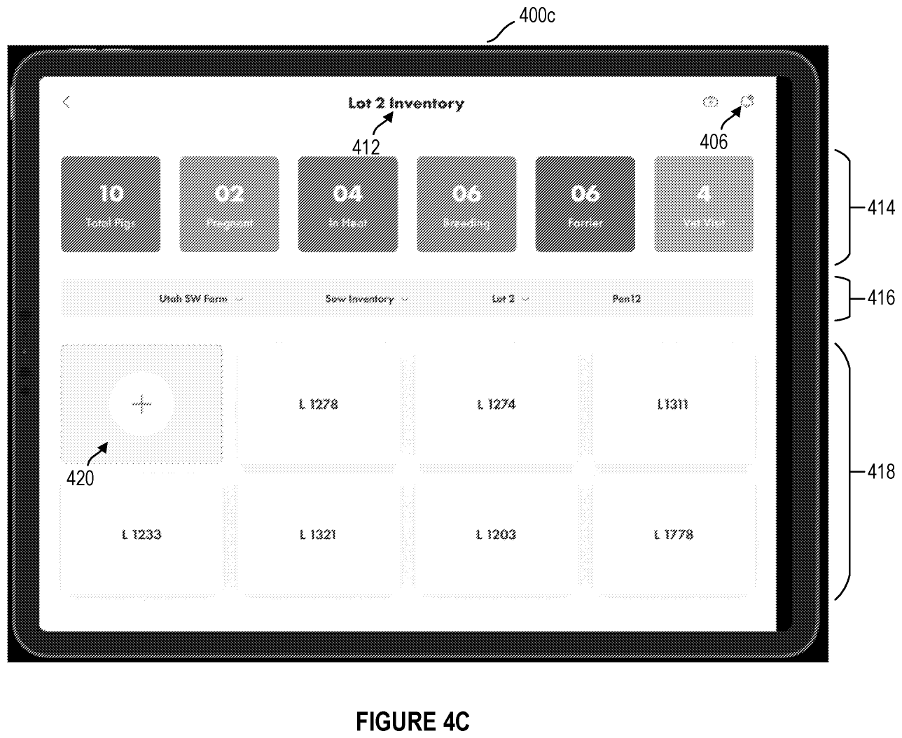

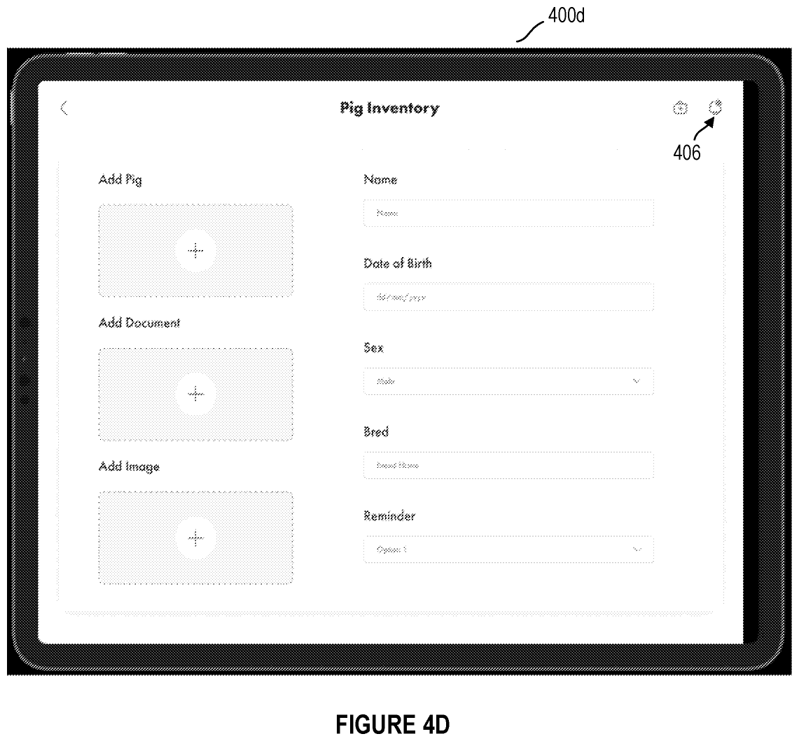

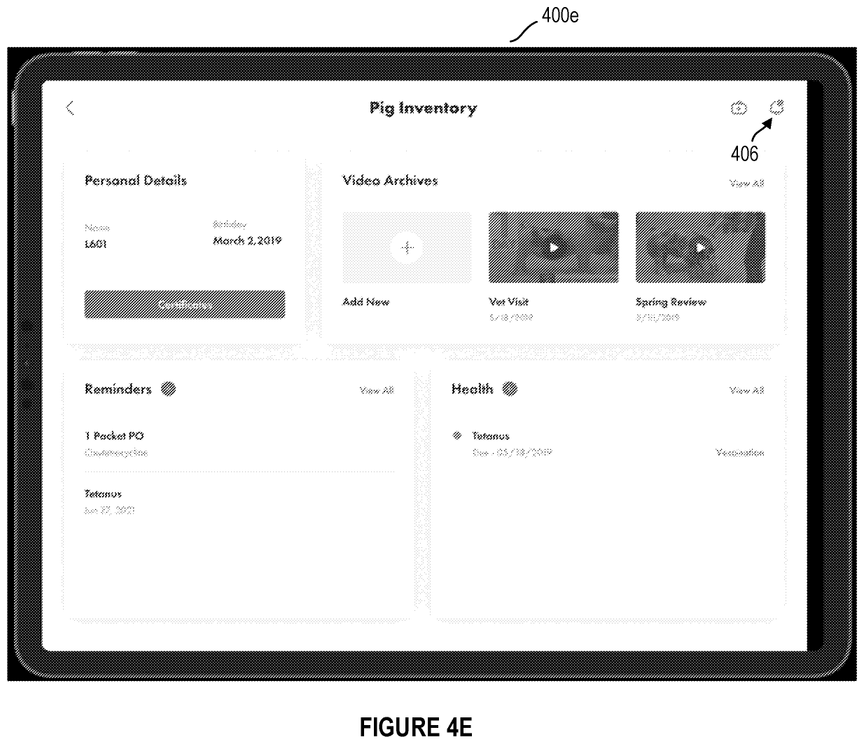

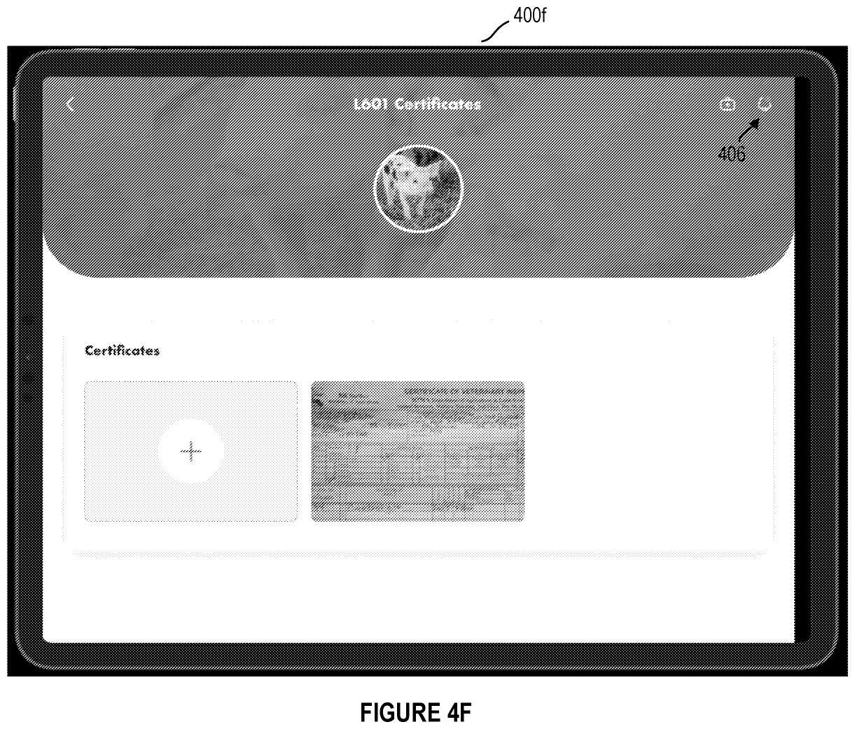

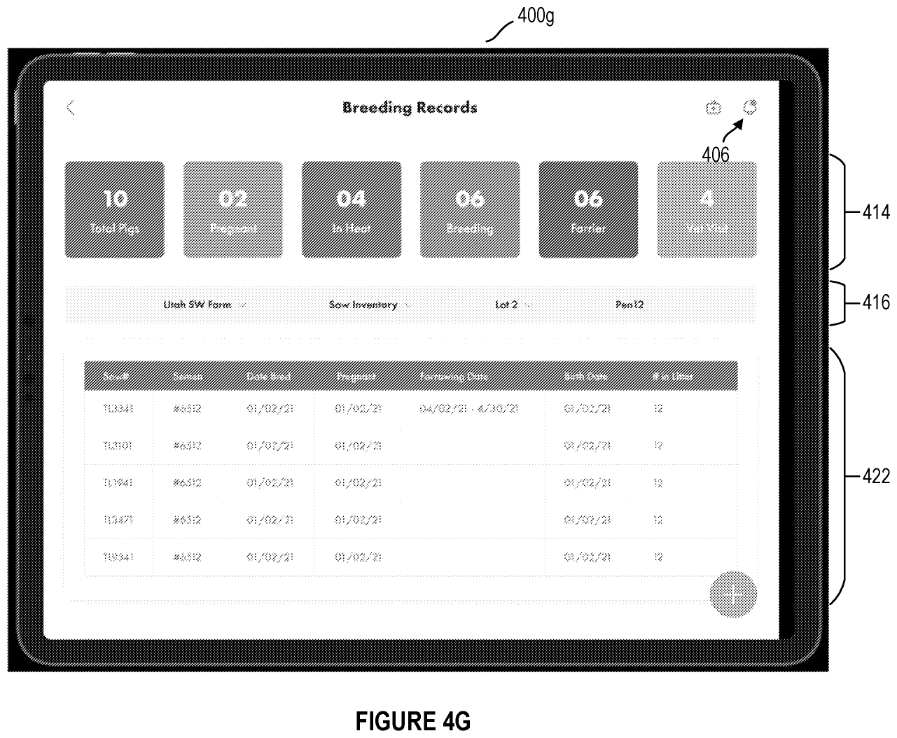

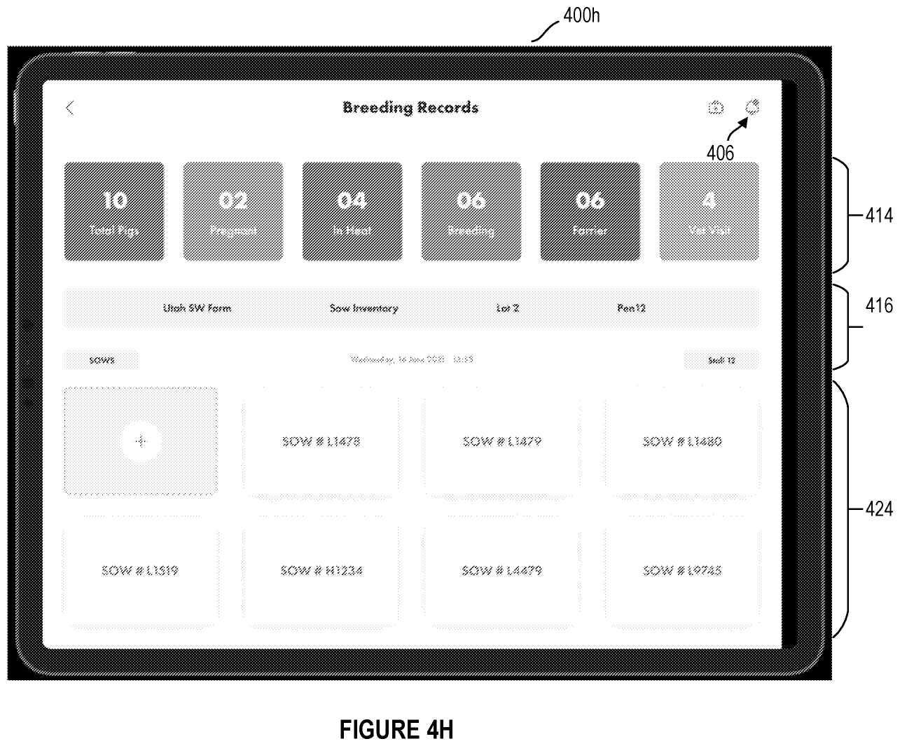

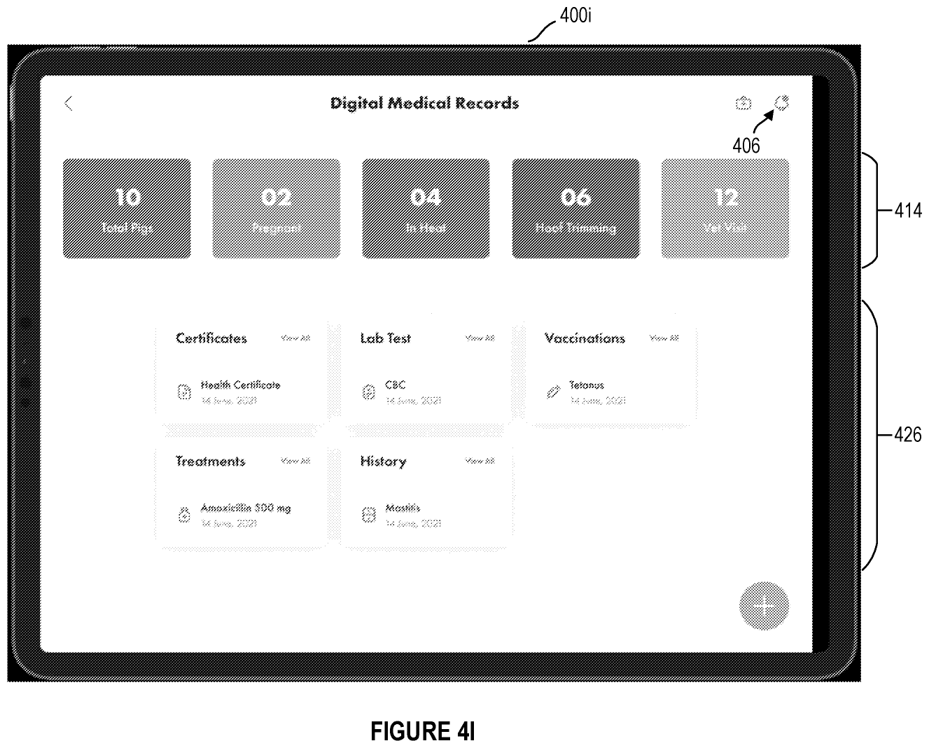

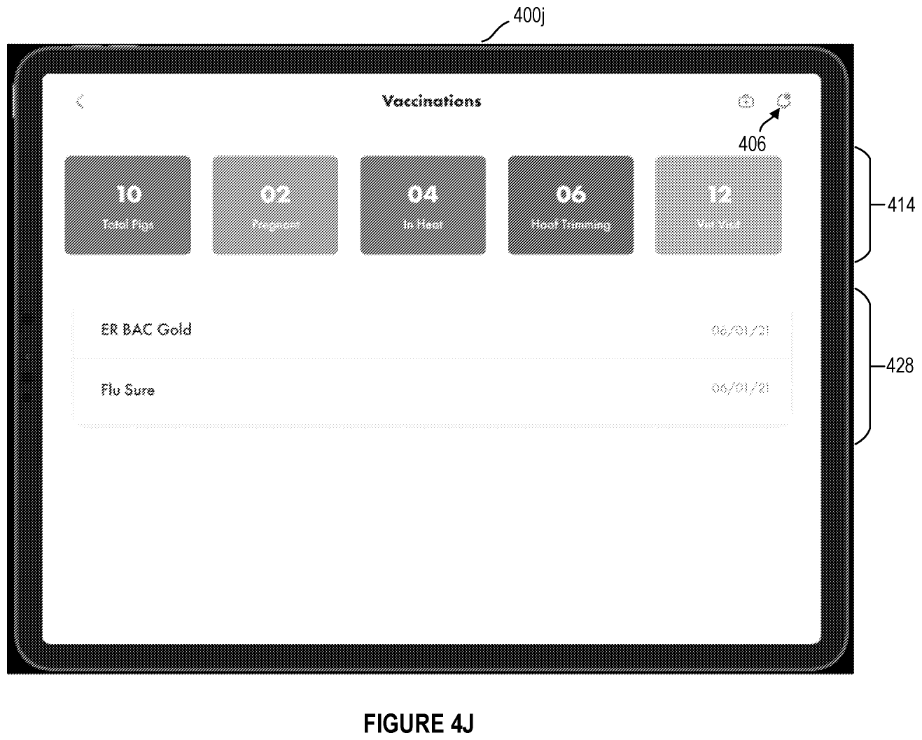

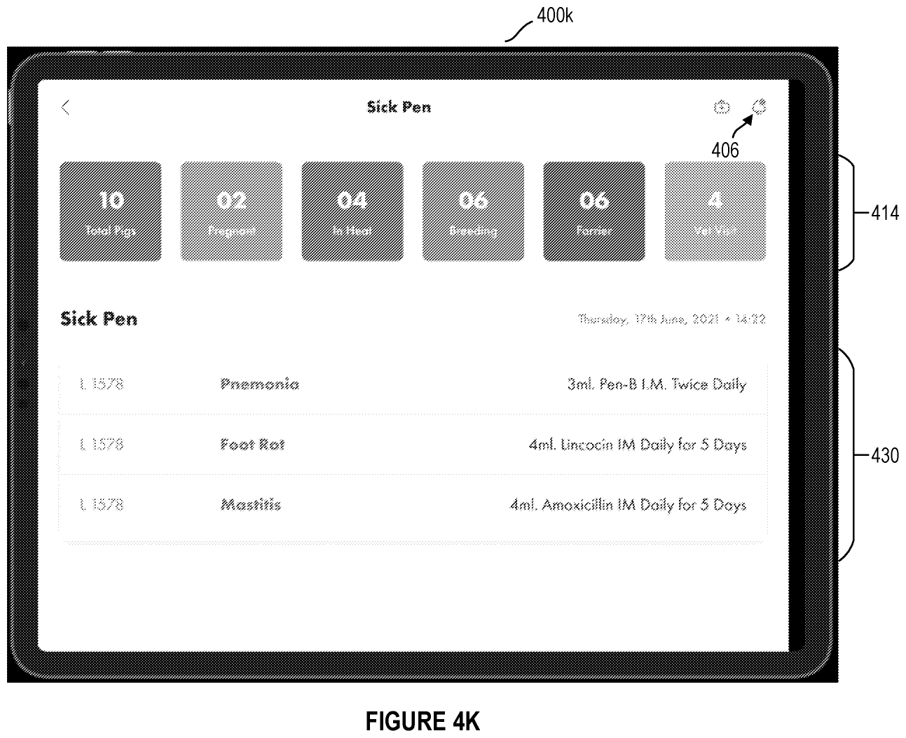

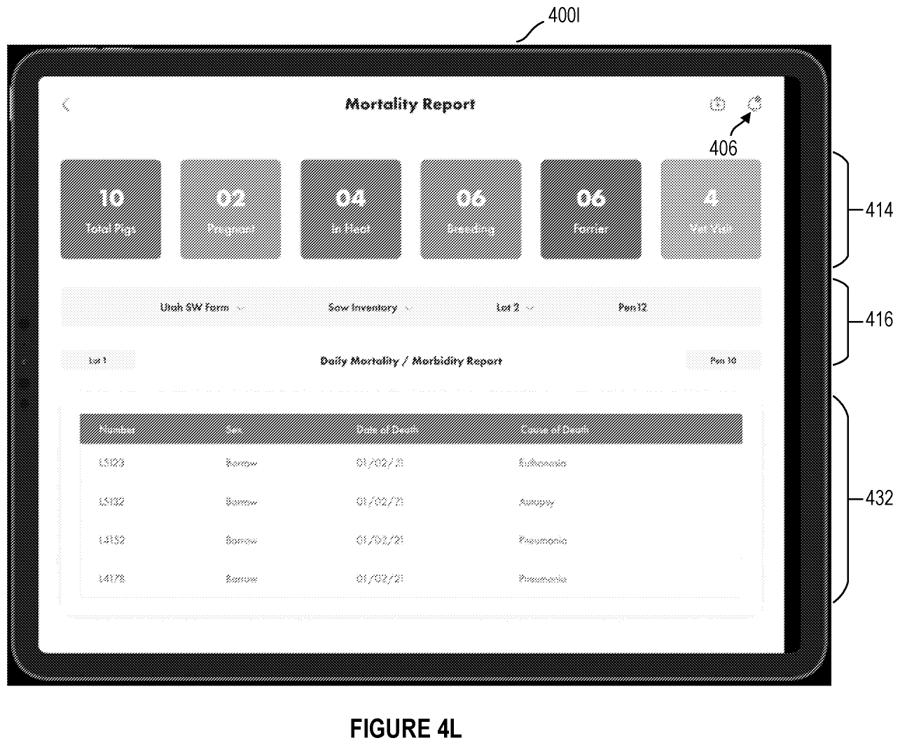

FIGS. 4A-4L illustrate example user interfaces 400 that may be generated for managing animal health, according to certain embodiments. For example, user interfaces 400 may be user interfaces that are generated by animal management application 210 stored on user devices 102. Although labeled user interface 400a, 400b, 400c, and the like, the user interfaces may be referred to generally as user interfaces 400.

Although particular user interfaces 400 are illustrated and described, this disclosure contemplates animal management application 210 generating these and/or other user interfaces. Although user interfaces 400 are illustrated and described as being arranged in a particular way, including particular content, or the like, user interfaces 400 and the particular arrangement and content of user interfaces 400 are for example purposes only.

Although primarily described in connection with an implementation for managing swine/pigs, embodiments of this disclosure may be used to manage any suitable type of animal, including, for example, pigs, cows, chickens, horses, sheep, or any other suitable type of animal.

As illustrated in FIG. 4A, user interface 400a includes an account identifier 402, which may identify one or more of an overall account (e.g., a corporate account associated with a farm), a particular user no, or the like.

User interface 400a may present a dashboard of menu options for managing animal health. For example, user interface 400a may include multiple selectable links 404a-404e (referred to generally as selectable links 404) that correspond to an associated feature and data set for managing animal health. Links 404 may operate as portals to particular data and feature sets associated with managing animal health.

In the illustrated example, a link 404a provides a link to the dashboard (e.g., user interface 400a). Link 404b provides a link to an animal inventory (e.g., “lot inventory”) user interface for providing data related to the inventory of animals being managed using animal management application 210. Link 404c provides a link to a breeding record user interface for some or all of the animals in the animal inventory. Link 404d provides a link to a medical record user interface for the animals in the animal inventory. Link 404e provides a link to a health reminders user interface for the animals in the animal inventory. Link 404f provides a link to a treatment inventory user interface for animals in the animal inventory. The treatment inventory also may be referred to as the sick pen, and may relate to animals that are undergoing treatment for one or more health problems. Link 404g provides a link to a mortality report user interface for animals in the animal inventory.

Selecting links 404 may cause animal management application 210 to access suitable animal records 112 stored on storage unit 106 and display, in a suitable user interface, information corresponding to the selected link 404. For example, in response to a user request, animal management application 210 may interact with animal management logic 310 to search animal records 112 stored on storage unit 106 to provide for display in one or more additional user interfaces 400 information responsive to the user request.

User interface 400a includes a notification 406 that indicates the availability of one or more reminders, such as reminders that may be further explained in the detailed health reminder page linked by link 404e. As will be seen throughout FIGS. 4B-4L, notification 406 may be persistently shown through some or all of interfaces 400, so that the associated reminders may be quickly accessed from any of a variety of user interfaces 400.

As illustrated in FIG. 4B, user interface 400b includes an animal inventory for a particular farm. User interface 400b includes a farm indicator 408, which in this example is identified as Utah—Farm 5 for Company A. User interface 400b includes animal inventory indicators 410, which may be grouped according to appropriate categories for the types of animals being managed using animal management application 210. In this example, an inventory of pigs at Utah—Farm 5 for Company A are divided into Lots 1-8, and user interface 400b indicates the number of pigs per lot.

Different types of animals may be grouped at farms in different ways. Taking pigs as an examples, a farm may include multiple lots, and each lot may include multiple pens, and each pen may include multiple stalls. Each lot may house a subset of the pigs for the farm, each pen may include a further subset of the pigs for a given lot, and each stall may include a further subset of the pigs for a given pen. Certain embodiments of this disclosure provide an ability to organize and view a pig inventory by farm, lot, pen, and stall. Although the particular terminology and division may be different, other types of animals may be similarly organized, if appropriate.

As illustrated in FIG. 4C, user interface 400c includes additional details regarding animal inventory. In particular, as indicated by title block 412 (Lot 2 Inventory), user interface 400c may include additional information related to the animal inventory of Lot 2. For example, from user interface 400b of FIG. 4B, a user may select the animal inventory indicator 410 for Lot 2, which may cause animal management application 210 to access via storage unit 106 animal inventory data for Lot 2 and display interface 400c that provides additional information for the animal inventory of Lot 2. User interface 400c provides a greater level of detail associated with a selected animal inventory.

In the illustrated example, user interface 400c includes a summary banner 414, a filter banner 416, and an animal list 418.

For quick reference, summary banner 414 includes, in various categories, a summary of the number of animals that fit into the identified categories. In this example, for the selected location (Utah SW Farm, females (Sow Inventory), Lot 2, Pen 12), the categories include the total number of pigs, the number of pregnant pigs, the number of pigs in heat, the number of pigs breeding, the number of pigs to see the farrier, and the number of pigs that are due for a visit by the veterinarian.

Filter banner 416 provides a number of fields that may be used to refine the list of animals shown to be included in the listed inventory and summarized in summary banner 414. In this example, the list of pigs is limited to the Utah SW Farm, females (Sow Inventory), Lot 2, Pen 12.

Animal list 418 includes identifiers of animals that are included in the inventory according to the selected criteria defined in filter banner 416. In certain embodiments, the animals may be tagged or otherwise labeled (e.g., branding, tattooing, etc.) with a label that includes this identifier.

User interface 400c also includes a selectable add-animal link 420, which may allow a user to add an animal (e.g., a pig) directly to the inventory for the group to which the information shown in user interface 400c corresponds (e.g., Utah SW Farm, Sow Inventory, Lot 2, Pen 12).

As illustrated in FIG. 4D, user interface flood may facilitate adding a pig to the inventory of pigs. For example, animal management application 210 may display user interface flood in response to receiving a user selection of add-animal link 420 from user interface 400c. User interface flood may allow a user to add a pig to the pig inventory. For example, user interface flood includes a number of fields for a user to enter data regarding a new pig, including the name, date of birth, sex, breed, any reminders, and the like. In the illustrated example, user interface flood includes links for adding an image of the pig being added, adding one or more documents associated with the pig being added, or adding another pig.

As illustrated in FIG. 4E, user interface 400e provides a view of detailed information for a particular pig in the pig inventory. User interface 400e may include personal details of the particular pig, such as a name (e.g., an ID), a birthday, and a link to any associated certificates (see, e.g., FIG. 4F, described below). User interface 400e may include one or more videos of the selected pig, along with a link for adding further videos. For example, the videos for the particular pig may include videos of one or more veterinarian visits, a periodic review, and the like. User interface 400e may include a reminders section that lists one or more reminders for the particular pig. Example reminders may include scheduled medications, scheduled vaccinations, scheduled veterinary visits, and the like.

As illustrated in FIG. 4F, user interface 400f may include one or more certificates for a particular pig, as well as a link for adding further certificates for the particular pig. An example certificate for the particular pig may include a certificate of veterinary inspection. In certain embodiments, user interface 400f for a particular pig may be accessed by selecting the certificates link on user interface 400e for the particular pig.

As illustrated in FIG. 4G, user interface 400g includes breeding records for a selected set of pigs of the pig inventory. For example, from user interface 400a of FIG. 4A, a user may select link 404c to a breeding record user interface 400g for some or all of the pigs in the pig inventory. Selecting link 404c from user interface 400a may cause animal management application 210 to access via storage unit 106 animal inventory data for a particular subdivision of the farm (Farm, Lot, Pen, and/or Stall) and display interface 400g that provides breeding records for that particular subdivision of the farm.

In the illustrated example, user interface 400g includes a summary banner 414, a filter banner 416, and an animal breeding list 422. Summary banner 414 may be similar to that described previously. Additionally, as described previously, filter banner 416 provides a number of fields that may be used to refine the list of animals shown to be included in the listed inventory and summarized in summary banner 414. In this example, the list of pigs is limited to the Utah SW Farm, females (Sow Inventory), Lot 2, Pen 12.

Animal breeding list 422 includes identifiers of sows in the selected, filtered inventory list, along with additional breeding details, such as the semen dose number, the date the sow was bred, the date the sow became pregnant, the farrowing date (or date range), the birthing date, and the number of piglets in the litter.

As illustrated in FIG. 4H, user interface 400h includes a different view of breeding records for a subset of the pigs in the pig inventory. In particular, a sow list 424 is limited to sows in stall 12, as further refined by filter banner 416. The sows identified in sow list 424 may be selected for further details on those particular sows, and additional sows may be added to the list using the “add” button.

As illustrated in FIG. 4I, user interface 400i includes digital medical records for a selected pig of the pig inventory. For example, from user interface 400a of FIG. 4A, a user may select link 404d to a digital medical record user interface 400i for some or all of the pigs in the pig inventory. Selecting link 404d from user interface 400a may cause animal management application 210 to access via storage unit 106 animal health data for a particular subdivision of the farm (Farm, Lot, Pen, and/or Stall) and display user interface 400i that provides digital medical records. In the illustrated example, user interface 400i displays health data 426 regarding digital medical records for a particular pig. Health data 426 includes a partial or complete list of certificates, lab tests, vaccinations, treatments, and medical history (e.g., illnesses). These lists of information may further include links to additional information regarding the listed items or additional items (e.g., a more complete historical event list).

As illustrated in FIG. 4J, user interface 400j includes a vaccine list 428 of vaccinations that have been administered to a particular pig, along with a date on which the vaccine was administered.

As illustrated in FIG. 4K, user interface 400k includes a sick pen list 430. Sick pen list 430 lists identifiers of pigs in the pig inventory that are being treated for an illness, along with an identification of the illness and the treatment being administered to treat the illness. From user interface 400a of FIG. 4A, a user may select link 404f to access user interface 400k that includes the sick pen list 430. Selecting link 404f from user interface 400a may cause animal management application 210 to access via storage unit 106 animal health data for a particular subdivision of the farm (Farm, Lot, Pen, and/or Stall) and display interface 400k that provides sick pen list 430.

As illustrated in FIG. 4L, user interface 400l includes mortality information for pigs in the pig inventory. For example, for the pigs filtered according to the fields defined in filter banner 416, mortality list 432 may list information regarding the pigs that have died. The information may include, for example, a pig identifier, the sex of the pig, the date of death, and the cause of death. From user interface 400a of FIG. 4A, a user may select link 404g to access user interface 400l that includes the mortality list 432. Selecting link 404g from user interface 400a may cause animal management application 210 to access via storage unit 106 animal health data for a particular subdivision of the farm (Farm, Lot, Pen, and/or Stall) and display interface 400l that provides mortality list 432.

FIG. 5 illustrates example digital animal records 112, according to certain embodiments. In the illustrated example, digital animal records 112 are stored using storage unit 106. Digital animal records 112 includes digital animal records 112a, digital animal records 112b, through digital animal records 112n, which may be referred to generally as digital animal records 112. Each set of digital animal records 112 may correspond to a different farm, a different corporate entity, or the like. In certain embodiments, digital animal records 112 for different entities (e.g., different corporate operations) may be stored in distinct storage units 106.

In this example, the animal data of digital animal records 112 (e.g., of digital animal records 112a) may be subdivided into animal inventory data 502 and animal health data 504. The particular subdivision of the data in digital animal records 112 that is illustrated and described is for example purposes only. Furthermore, although described separately, some of the data may overlap. Additionally, the digital animal records 112 may be organized in storage unit 106 in any suitable manner.

Animal inventory data 502 may include information for identifying and locating the animals, as well as certain factual information about the animals. For example, animal inventory data 502 may include identifiers for identifying the animals. As a particular example, an identifier of an animal may be a string of alphanumeric characters assigned to the animal. Additionally or alternatively, the identifier may be a code, such as a machine-readable image file or a physical representation thereof, examples of which include a quick response (QR) code and/or a barcode. In certain scenarios, the animal identifier may be originally assigned to the source animal when the source animal is entered into system 100 (e.g., into digital animal records 112). Although this disclosure contemplates any suitable component providing the animal identifier, in certain embodiments, processing system 104 (e.g., animal management logic 310) generates the animal identifier, such as when a user submits the form shown on interface flood. The animal might or might not be tagged, branded, or otherwise physically labeled with the identifier. As another example, animal inventory data 502 may include biographical information for the animals. Such biographical information may include birthday, sex, and any other suitable biographical information. As another example, animal inventory data 502 may include location information identifying a location of the animal. The type of location information may depend on the type of animal as well as the manner in which a particular entity organizes animals. In a particular example of swine, the location information may include a suitable combination of Farm, Lot, Pen, and Stall.

Animal health data 504 may include information regarding the health of the animals being managed using system 100. For example, animal health data 504 may include medical records for the animals. As another example, animal health data 504 may include breeding information for the animals. As another example, animal health data 504 may include vaccination records for the animals. As another example, animal health data 504 may include medications for the animals. As another example, animal health data 504 may include historical, current, or future health treatments for the animals. As another example, animal health data 504 may include mortality information for the animals. As another example, animal health data 504 may include health reminders for the animals. Such health reminders may include reminders to administer certain medications, reminders for veterinarian visits, and the like.

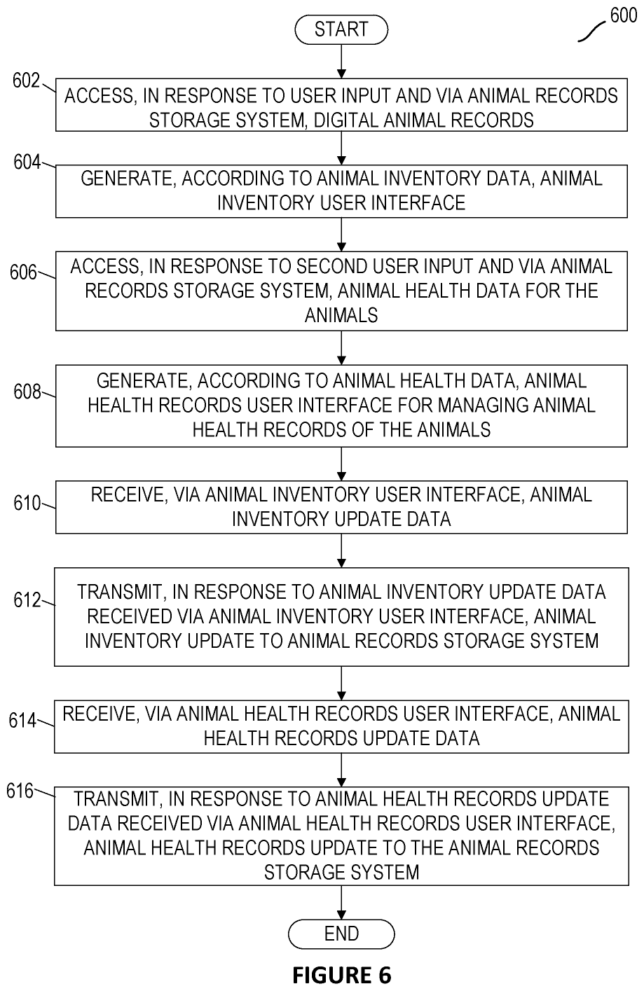

FIG. 6 illustrates an example method 600 for managing digital animal records for managing animal health, according to certain embodiments. In certain embodiments, the operations of method 600 may be performed by animal management application 210 running on user device 102. In certain embodiments, the animals being managed using system 100 may be cows or pigs, however, this disclosure contemplates managing any suitable type or types of animals using system 100 and method 600.

At step 602, animal management application 210 may access, in response to user input using user device 102 and via an animal records storage system (e.g., storage unit 106), digital animal records 112 for a plurality of animals. Digital animal records 112 may store animal data for the animals, and the animal data may include animal inventory data and animal health data.

At step 604, animal management application 210 may generate, according to the animal inventory data, an animal inventory user interface. The animal inventory user interface may include identifying information of at least a first portion of the animals being managed using system 100. In certain embodiments, animal management application 210 may receive, via the animal inventory user interface, a selection of a subset of the animals and generate an updated animal inventory user interface according to the selection of the subset of animals. For example, using the fields of a filter banner 416, a user may specify to see some or all of the animal inventory data for a particular subset of the animals. In response to the filter request, animal management application 210 may communicate with animal management logic 310 to retrieve the requested animal inventory data and display an animal inventory user interface that includes the requested animal inventory data.

In certain embodiments, the animal inventory user interface generated by animal management application 210 includes a link for adding a new animal to the inventory of managed animals. In such an example, animal management application 210 may be configured to receive animal inventory data for the new animal and transmit the animal inventory data for the new animal for storage in the animal records storage system (e.g., in digital animal records 112 of storage unit 106).

At step 606, animal management application 210 may access, in response to user input from user device 102 and via the animal records storage system (e.g., storage unit 106), animal health data for the animals.

At step 608, animal management application 210 may generate, according to the animal health data, an animal health records user interface for managing the animal health records of the animals. The animal health records user interface may include medical records of at least a portion of the animals.

At step 610, animal management application 210 may receive, via the animal inventory user interface displayed on user device 102, animal inventory update data, and at step 612, animal management application 210 may transmit, in response to the animal inventory update data received via the animal inventory user interface, an animal inventory update to the animal records storage system (e.g., storage unit 106) for updating digital animal records 112 (e.g., animal inventory data).

At step 614, animal management application 210 may receive, via the animal health records user interface displayed on user device 102, animal health records update data; and at step 616, animal management application 210 may transmit, in response to the animal health records update data received via the animal health records user interface, an animal health records update to the animal records storage system (e.g., storage unit 106) for updating digital animal records 112 (e.g., animal health data).

In certain embodiments, animal management application 210 may generate a dashboard user interface screen that includes selectable links. Each selectable link may correspond to an associated feature and data set for managing animal health of the managed animal inventory. For example, a first selectable link may include a link to the animal inventory user interface. As another example, a second selectable link may include a link to a breeding records user interface. As another example, a third selectable link may include a link to a medical records user interface. As another example, a fourth selectable link may include a link to a health reminders user interface. As another example, a fifth selectable link may include a link to a treatment user interface. As another example, a sixth selectable link may include a link to a mortality report user interface.

In certain embodiments, animal management application 210 is configured to access, via the animal records storage system (e.g., storage unit 106), breeding information for a subset of the animals, and to generating, according to the breeding information, a breeding user interface. As examples, the breeding user interface may include breeding information for the subset of animals or the breeding user interface may include breeding details for a particular animal of the subset of animals.

In certain embodiments, animal management application 210 is configured to access, via the animal records storage system (e.g., storage unit 106), mortality information of the animals, and to generate, according to the mortality information, a mortality user interface summarizing the mortality information for the animals.

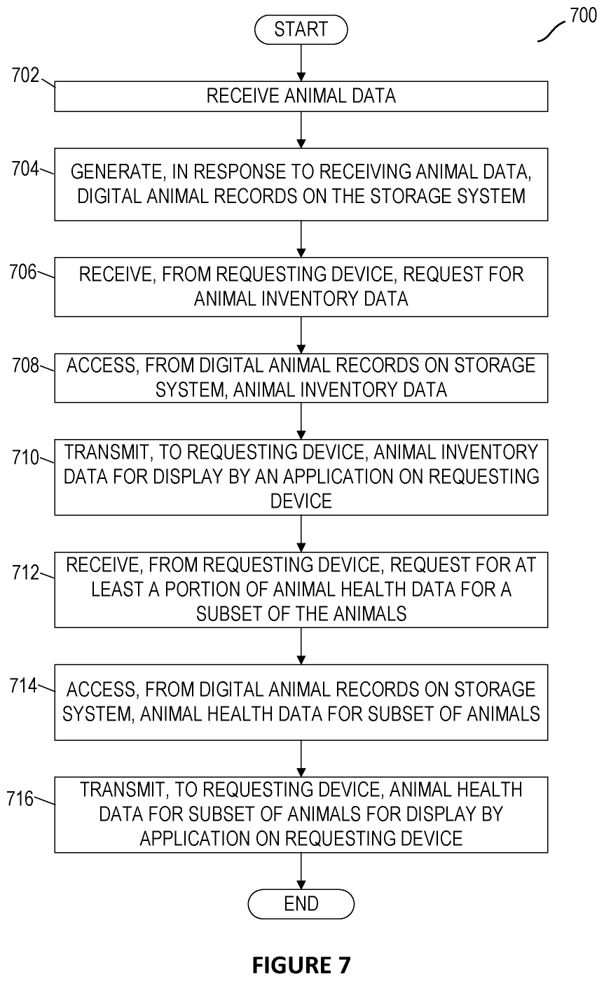

FIG. 7 illustrates an example method 700 for managing digital animal records for managing animal health, according to certain embodiments. In certain embodiments, the operations of method 700 may be performed by animal management logic 310 running on processing system 104. In certain embodiments, the animals being managed using system 100 may be cows or pigs, however, this disclosure contemplates managing any suitable type or types of animals using system 100 and method 700.

At step 702, animal management logic 310 may receive animal data for a plurality of animals. The animal data may include animal inventory data and animal health data. At step 704, animal management logic 310 may generate, in response to receiving the animal data for the plurality of animals, digital animal records 112 on the storage system (e.g., storage unit 106). Digital animal records 112 may include the animal data for the plurality of animals.

At step 706, animal management logic 310 may receive, from a requesting user device 102, a request for the animal inventory data for the plurality of animals. At step 708, animal management logic 310 may access, from digital animal records 112 on the storage system (e.g., storage unit 106), the animal inventory data for the plurality of animals, and at step 710, animal management logic 310 may transmit, to the requesting user device 102, the animal inventory data for display by an application (e.g., animal management application 210) running on the requesting user device 102. In certain embodiments, the animal inventory data includes the following information for the managed inventory of animals: an animal identifier; animal type; and location information.

At step 712, animal management logic 310 may receive, from a requesting user device 102, a request for at least a portion of the animal health data for a subset of the animals. Of course the user device 102 from which the request received at step 712 is received may be the same or different as the user device 102 from which the request received at step 706 is received. At step 714, animal management logic 310 may access, from digital animal records 112 on the storage system (e.g., storage unit 106), the animal health data for the subset of the animals, and at step 716, animal management logic 310 may transmit, to the requesting user device 102, the animal health data for the subset of the animals for display by an application (e.g., animal management application 210) running on the requesting user device 102. In certain embodiments, the animal health data includes the following information for the managed inventory of animals: digital medical records; vaccination information; breeding information; treatment information; and mortality information. In certain embodiments, the animal health data includes a schedule of health events and animal management logic 310 is configured to communicate a reminder of a health event to an application (e.g., animal management application 210) running on a requesting user device 102.

Although the present disclosure describes or illustrates particular operations as occurring in a particular order, the present disclosure contemplates any suitable operations occurring in any suitable order. Moreover, the present disclosure contemplates any suitable operations being repeated one or more times in any suitable order. Although the present disclosure describes or illustrates particular operations as occurring in sequence, the present disclosure contemplates any suitable operations occurring at substantially the same time, where appropriate. Any suitable operation or sequence of operations described or illustrated herein may be interrupted, suspended, or otherwise controlled by another process, such as an operating system or kernel, where appropriate. The acts can operate in an operating system environment or as stand-alone routines occupying all or a substantial part of the system processing.

Certain embodiments may provide none, some, or all of the following technical advantages.

Certain embodiments provide a highly intuitive user interface for viewing, sharing, updating, or otherwise interacting with animal data stored in digital animal records, including animal inventory data and animal health data. For example, through a software application running on a mobile device, farmers and other workers associated with a livestock operation may enter animal data for one or more groups of animals being raised by the livestock operation to establish digital data records for those animals. Those digital data records include animal inventory data and animal health data. The software application may provide a variety of interfaces for interacting with the digital data records to facilitate managing the health of the animals being raised by the livestock operation.

Certain embodiments may save time by reducing or eliminating hand-written health records through a partial or complete digital solution. Certain embodiments may reduce costs by allowing decision makers to use real-time data to make timely, potentially improved management decisions. Certain embodiments allow farmers and associated users to communicate with a veterinarian from virtually anywhere at any time. Certain embodiments may improve animal health. Certain embodiments may cut veterinary costs through better routine health programs. Certain embodiments provide a veterinary electronic medical record for an animal or group of animals.

Through the user interfaces implemented according to certain embodiments of this disclosure, certain embodiments are able to display a limited set of information in an organized manner, resulting in an improved user interface, to facilitate managing the health of the livestock of an organization. User interfaces provided by certain embodiments of this disclosure may allow a user to more quickly access data and applications in electronic devices, improving the speed of a user's navigation through various views and windows.

Example embodiments of this disclosure are summarized here. Other embodiments can also be understood from the entirety of the specification as well as the claims filed herein.

Example 1: A non-transitory computer-readable storage medium stores a program for execution by one or more processors. The program includes instructions for accessing, in response to a first user input and via an animal records storage system, digital animal records for animals. The digital animal records include animal data for the animals, the animal data including animal inventory data and animal health data. The program includes instructions for generating, according to the animal inventory data, an animal inventory user interface that includes identifying information of at least a first portion of the animals. The program includes instructions for accessing, in response to a second user input and via the animal records storage system, the animal health data for the animals and generating, according to the animal health data, an animal health records user interface for managing the animal health records of the animals. The animal health records user interface includes medical records of at least a second portion of the animals. The program includes instructions for receiving, via the animal inventory user interface, animal inventory update data and transmitting, in response to the animal inventory update data, an animal inventory update to the animal records storage system for updating the animal inventory data. The program includes instructions for receiving, via the animal health records user interface, animal health records update data and transmitting, in response to the animal health records update data, an animal health records update to the animal records storage system for updating the animal health data.

Example 2: The non-transitory computer-readable medium of Example 1, where the program further includes instructions for: receiving, via the animal inventory user interface, a selection of a subset of the animals; and generating an updated animal inventory user interface according to the selection of the subset of the animals.

Example 3: The non-transitory computer-readable medium of any one of Examples 1-2, where: the animal inventory user interface comprises a link for adding a new animal to the animals; and the program further includes instructions for: receiving animal inventory data for the new animal; and transmitting the animal inventory data for the new animal for storage in the animal records storage system.

Example 4: The non-transitory computer-readable medium of any one of Examples 1-3, where the animals are cows or pigs.

Example 5: The non-transitory computer-readable medium of any one of Examples 1-4, where the program further includes instructions for generating a dashboard user interface screen that includes selectable links, each selectable link corresponding to an associated feature and data set for managing animal health of the animals.

Example 6: The non-transitory computer-readable storage medium of Example 5, where: a first selectable link of the selectable links includes a link to the animal inventory user interface; a second selectable link of the selectable links includes a link to a breeding records user interface; a third selectable link of the selectable links includes a link to a medical records user interface; a fourth selectable link of the selectable links includes a link to a health reminders user interface; a fifth selectable link of the selectable links includes a link to a treatment user interface; and a sixth selectable link of the selectable links includes a link to a mortality report user interface.

Example 7: The non-transitory computer-readable storage medium of any one of Examples 1-6, where the program further includes instructions for: accessing, via the animal records storage system, breeding information for a subset of the animals; and generating, according to the breeding information, a breeding user interface.

Example 8: The non-transitory computer-readable storage medium of Example 7, where: the breeding user interface comprises breeding information for the subset of the animals; or the breeding user interface comprises breeding details for a particular animal of the subset of the animals.

Example 9: The non-transitory computer-readable storage medium of any one of Examples 1-8, where the program further includes instructions for: accessing, via the animal records storage system, mortality information of the animals; and generating, according to the mortality information, a mortality user interface summarizing the mortality information for the animals.

Example 10: A system includes a storage system, one or more processors, and a non-transitory computer-readable storage medium storing a program to be executed by the one or more processors. The program includes instructions for receiving animal data for animals, the animal data including animal inventory data and animal health data, and for generating, in response to receiving the animal data, digital animal records on the storage system, the digital animal records including the animal data. The program includes instructions for receiving, from a first requesting device, a request for the animal inventory data for the animals; accessing, from digital animal records on the storage system, the animal inventory data for the animals; and transmitting, to the first requesting device, the animal inventory data for display by an application on the first requesting device. The program includes instructions for receiving, from a second requesting device, a request for at least a portion of the animal health data for a subset of the animals; accessing, from the digital animal records on the storage system, the animal health data for the subset of the animals; and transmitting, to the second requesting device, the animal health data for the subset of the animals for display by an application on the second requesting device.

Example 11: The system of Example 10, where the first requesting device and the second requesting device are a same device.

Example 12: The system of any one of Examples 10-11, where: the animal health data includes a schedule of health events; and the program further includes instructions for communicating a reminder of a health event to an application on a third requesting device.

Example 13: The system of any one of Examples 10-12, where the animal inventory data includes the following information for the animals: an animal identifier; animal type; and location information.

Example 14: The system of any one of Examples 10-13, where the animal health data includes the following information for the animals: digital medical records; vaccination information; breeding information; treatment information; and mortality information.

Example 15: A method includes accessing, in response to a first user input and via a storage system, digital animal records that include animal data (including inventory and health data) for animals; and generating, according to the inventory data, an inventory user interface (UI) that includes identifying information of a first portion of the animals. The method includes accessing, in response to a second user input and via the storage system, the health data and generating, according to the health data, a health records UI for managing the health records and that includes medical records of a second portion of the animals. The method includes receiving, via the inventory UI, inventory update data and, in response, transmitting an inventory update to the storage system. The method includes receiving, via the health records UI, health records update data and, in response, transmitting a health records update to the storage system.

Example 16: The method of Example 15, further including: receiving, via the animal inventory user interface, a selection of a subset of the animals; and generating an updated animal inventory user interface according to the selection of the subset of the animals.

Example 17: The method of any one of Examples 15-16, where: the animal inventory user interface includes a link for adding a new animal to the animals; and the method further includes: receiving animal inventory data for the new animal; and transmitting the animal inventory data for the new animal for storage in the animal records storage system.

Example 18: The method of any one of Examples 15-17, where the method further includes generating a dashboard user interface screen that includes selectable links, each selectable link corresponding to an associated feature and data set for managing animal health of the animals.

Example 19: The method of Example 18, where: a first selectable link of the selectable links includes a link to the animal inventory user interface; a second selectable link of the selectable links includes a link to a breeding records user interface; a third selectable link of the selectable links includes a link to a medical records user interface; a fourth selectable link of the selectable links includes a link to a health reminders user interface; a fifth selectable link of the selectable links includes a link to a treatment user interface; and a sixth selectable link of the selectable links includes a link to a mortality report user interface.

Example 20: The method of any one of Examples 15-19, further including: accessing, via the animal records storage system, breeding information for a subset of the animals; and generating, according to the breeding information, a breeding user interface.

In certain embodiments, a system includes one or more processors and a non-transitory computer-readable storage medium storing a program to be executed by the processor. The program includes instructions for: generating a user interface comprising fields for managing animal health associated with one or more animals; generate a digital data record for the one or more animals, the digital data record comprising health records associated with the one or more animals; and causing the digital data record to be stored.

In certain embodiments, a non-transitory computer-readable storage medium stores a program to be executed by one or more processors. The program includes instructions for: generating a user interface comprising fields for managing animal health associated with one or more animals; generating a digital data record for the one or more animals, the digital data record comprising health records associated with the one or more animals; and causing the digital data record to be stored.

In certain embodiments, a method includes: generating a user interface comprising fields for managing animal health associated with one or more animals; generating a digital data record for the one or more animals, the digital data record comprising health records associated with the one or more animals; and causing the digital data record to be stored.

An example processing system, according to certain embodiments of this disclosure, may be configured to perform methods/operations described in this disclosure, and may be installed in a host device. The processing system may include a processor, a memory, and interfaces. The processor may be any component or collection of components adapted to perform computations and/or other processing related tasks, and the memory may be any component or collection of components adapted to store programming and/or instructions for execution by processor. In an embodiment, the memory includes a non-transitory computer readable medium. The computer-readable non-transitory media includes all types of computer readable media, including magnetic storage media, optical storage media, and solid-state storage media and specifically excludes signals. It should be understood that the software can be installed in and sold with the device. Alternatively, the software can be obtained and loaded into the device, including obtaining the software via a disc medium or from any manner of network or distribution system, including, for example, from a server owned by the software creator or from a server not owned but used by the software creator. The software can be stored on a server for distribution over the Internet, for example.

While this disclosure has been described with reference to illustrative embodiments, this description is not intended to be construed in a limiting sense. Various modifications and combinations of the illustrative embodiments, as well as other embodiments of the disclosure, will be apparent to persons skilled in the art upon reference to the description. It is therefore intended that the appended claims encompass any such modifications or embodiments.

What is claimed is:

1. A non-transitory computer-readable storage medium storing a program for execution by one or more processors, the program including instructions for:
accessing, in response to a first user input and via an animal records storage system, digital animal records for a plurality of animals, the digital animal records comprising animal data for the plurality of animals, the animal data comprising animal inventory data and animal health data;
generating, according to the animal inventory data, an animal inventory user interface, wherein the animal inventory user interface comprises identifying information of at least a first portion of the plurality of animals;
accessing, in response to a second user input and via the animal records storage system, the animal health data for the plurality of animals;
generating, according to the animal health data, an animal health records user interface for managing the animal health records of the plurality of animals, wherein the animal health records user interface comprises medical records of at least a second portion of the plurality of animals;
receiving, via the animal inventory user interface, animal inventory update data;
transmitting, in response to the animal inventory update data received via the animal inventory user interface, an animal inventory update to the animal records storage system for updating the animal inventory data;
receiving, via the animal health records user interface, animal health records update data; and
transmitting, in response to the animal health records update data received via the animal health records user interface, an animal health records update to the animal records storage system for updating the animal health data.

2. The non-transitory computer-readable medium of claim 1, wherein the program further includes instructions for:
receiving, via the animal inventory user interface, a selection of a subset of the plurality of animals; and
generating an updated animal inventory user interface according to the selection of the subset of the plurality of animals.

3. The non-transitory computer-readable medium of claim 1, wherein:
the animal inventory user interface comprises a link for adding a new animal to the plurality of animals; and
the program further includes instructions for:
receiving animal inventory data for the new animal; and
transmitting the animal inventory data for the new animal for storage in the animal records storage system.

4. The non-transitory computer-readable medium of claim 1, wherein the animals are cows or pigs.

5. The non-transitory computer-readable medium of claim 1, wherein the program further includes instructions for generating a dashboard user interface screen that comprises a plurality of selectable links, each selectable link of the plurality of selectable links corresponding to an associated feature and data set for managing animal health of the plurality of animals.

6. The non-transitory computer-readable storage medium of claim 5, wherein:
a first selectable link of the plurality of selectable links comprises a link to the animal inventory user interface;
a second selectable link of the plurality of selectable links comprises a link to a breeding records user interface;
a third selectable link of the plurality of selectable links comprises a link to a medical records user interface;
a fourth selectable link of the plurality of selectable links comprises a link to a health reminders user interface;
a fifth selectable link of the plurality of selectable links comprises a link to a treatment user interface; and
a sixth selectable link of the plurality of selectable links comprises a link to a mortality report user interface.

7. The non-transitory computer-readable storage medium of claim 1, wherein the program further includes instructions for:
accessing, via the animal records storage system, breeding information for a subset of the plurality of animals; and
generating, according to the breeding information, a breeding user interface.

8. The non-transitory computer-readable storage medium of claim 7, wherein:
the breeding user interface comprises breeding information for the subset of the plurality of animals; or
the breeding user interface comprises breeding details for a particular animal of the subset of the plurality of animals.

9. The non-transitory computer-readable storage medium of claim 1, wherein the program further includes instructions for:
accessing, via the animal records storage system, mortality information of the plurality of animals; and
generating, according to the mortality information, a mortality user interface summarizing the mortality information for the plurality of animals.

10. A system, comprising:
a storage system;
one or more processors;
a non-transitory computer-readable storage medium storing a program to be executed by the one or more processors, the program including instructions for:
receiving animal data for a plurality of animals, the animal data comprising animal inventory data and animal health data;
generating, in response to receiving the animal data for the plurality of animals, digital animal records on the storage system, the digital animal records comprising the animal data for the plurality of animals;
receiving, from a first requesting device, a request for the animal inventory data for the plurality of animals;
accessing, from digital animal records on the storage system, the animal inventory data for the plurality of animals;
transmitting, to the first requesting device, the animal inventory data for display by an application on the first requesting device;
receiving, from a second requesting device, a request for at least a portion of the animal health data for a subset of the plurality of animals;
accessing, from the digital animal records on the storage system, the animal health data for the subset of the plurality of animals; and
transmitting, to the second requesting device, the animal health data for the subset of the plurality of animals for display by an application on the second requesting device.

11. The system of claim 10, wherein the first requesting device and the second requesting device are a same device.

12. The system of claim 10, wherein:
the animal health data comprises a schedule of health events; and
the program further includes instructions for communicating a reminder of a health event to an application on a third requesting device.

13. The system of claim 10, wherein the animal inventory data comprises the following information for the plurality of animals:
an animal identifier;
animal type; and
location information.

14. The system of claim 10, wherein the animal health data comprises the following information for the plurality of animals:
digital medical records;
vaccination information;
breeding information;
treatment information; and
mortality information.

15. A method, comprising:
accessing, in response to a first user input and via an animal records storage system, digital animal records for a plurality of animals, the digital animal records comprising animal data for the plurality of animals, the animal data comprising animal inventory data and animal health data;
generating, according to the animal inventory data, an animal inventory user interface, wherein the animal inventory user interface comprises identifying information of at least a first portion of the plurality of animals;
accessing, in response to a second user input and via the animal records storage system, the animal health data for the plurality of animals;
generating, according to the animal health data, an animal health records user interface for managing the animal health records of the plurality of animals, wherein the animal health records user interface comprises medical records of at least a second portion of the plurality of animals;
receiving, via the animal inventory user interface, animal inventory update data;
transmitting, in response to the animal inventory update data received via the animal inventory user interface, an animal inventory update to the animal records storage system for updating the animal inventory data;
receiving, via the animal health records user interface, animal health records update data; and
transmitting, in response to the animal health records update data received via the animal health records user interface, an animal health records update to the animal records storage system for updating the animal health data.

16. The method of claim 15, further comprising:
receiving, via the animal inventory user interface, a selection of a subset of the plurality of animals; and
generating an updated animal inventory user interface according to the selection of the subset of the plurality of animals.

17. The method of claim 15, wherein:
the animal inventory user interface comprises a link for adding a new animal to the plurality of animals; and
the method further comprises:
receiving animal inventory data for the new animal; and
transmitting the animal inventory data for the new animal for storage in the animal records storage system.

18. The method of claim 15, wherein the method further comprises generating a dashboard user interface screen that comprises a plurality of selectable links, each selectable link of the plurality of selectable links corresponding to an associated feature and data set for managing animal health of the plurality of animals.

19. The method of claim 18, wherein:
a first selectable link of the plurality of selectable links comprises a link to the animal inventory user interface;
a second selectable link of the plurality of selectable links comprises a link to a breeding records user interface;
a third selectable link of the plurality of selectable links comprises a link to a medical records user interface;
a fourth selectable link of the plurality of selectable links comprises a link to a health reminders user interface;
a fifth selectable link of the plurality of selectable links comprises a link to a treatment user interface; and
a sixth selectable link of the plurality of selectable links comprises a link to a mortality report user interface.

20. The method of claim 15, further comprising:
accessing, via the animal records storage system, breeding information for a subset of the plurality of animals; and
generating, according to the breeding information, a breeding user interface.
